(19) United States
(12) Patent Application Publication
(10) Pub. No.: US 2025/0177269 A1
(43) Pub. Date: Jun. 5, 2025

(54) COMPOSITION FOR PROMOTING HAIR SPROUTING OR HAIR GROWTH CONTAINING A THYMOL ESTER-BASED COMPOUND

(71) Applicant: AMOREPACIFIC CORPORATION, Seoul (KR)

(72) Inventors: Suna KIM, Yongin-si (KR); Gyusang Jeong, Yongin-si (KR); Seunghyun Shin, Yongin-si (KR); Hyoung June Kim, Yongin-si (KR); Hyunjung Choi, Yongin-si (KR)

(73) Assignee: AMOREPACIFIC CORPORATION, Seoul (KR)

(21) Appl. No.: 18/968,486

(22) Filed: Dec. 4, 2024

(30) Foreign Application Priority Data
Dec. 5, 2023 (KR) 10-2023-0174413

(51) Int. Cl. A61K 8/37; A61K 31/235; A61P 17/14; A61Q 7/00

(52) U.S. Cl. CPC A61K 8/37; A61K 31/235; A61P 17/14; A61Q 7/00

(57) ABSTRACT

A composition for promoting hair sprouting or hair growth, including 3,4,5-trimethoxycinnamate thymol ester or a stereoisomer, pharmaceutically acceptable salt, hydrate, or solvate thereof as an active ingredient is disclosed. The composition can exhibit a significant effect on a subject in need of hair loss treatment or hair sprouting promotion and hair growth.

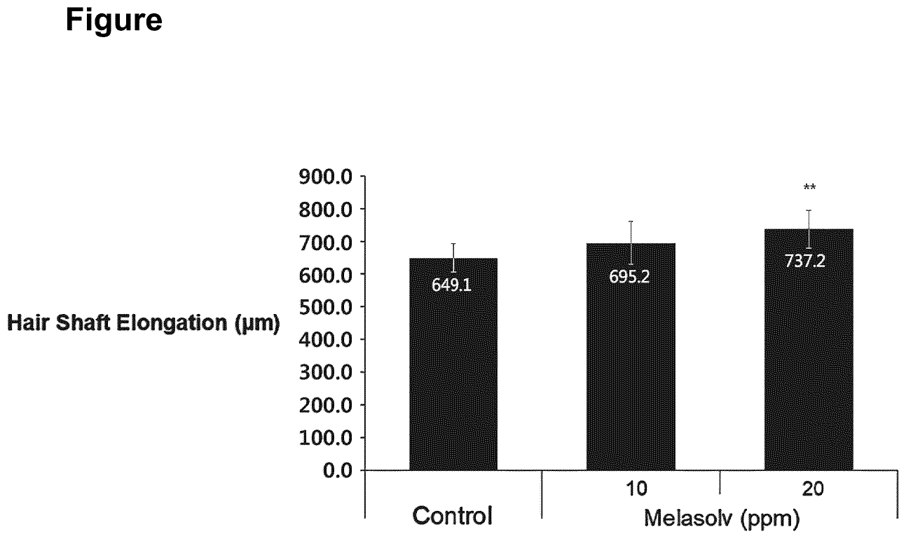

### CROSS REFERENCE TO RELATED APPLICATION

This application claims priority to Korea Patent Application No. 10-2023-0174413 filed on Dec. 5, 2023, the entire contents of which are incorporated herein by reference.

### BACKGROUND OF THE INVENTION

### Field of the Invention

Disclosed herein is a composition for promoting hair sprouting or hair growth, including 3,4,5-trimethoxycinnamate thymol ester or a stereoisomer, pharmaceutically acceptable salt, hydrate, or solvate thereof as an active ingredient.

### Description of the Related Art

Human hair primarily serves the function of protecting the scalp from external stimuli such as ultraviolet rays, and also serves the aesthetic function of expressing an individual's external image.

However, in modern society, hair loss frequently occurs due to natural factors such as environmental pollution and strong ultraviolet rays, as well as physiological factors such as stress and hormonal imbalance. Unlike in the past when it was limited to adult men, hair loss occurs regardless of gender or age, and thus, the need for materials for prevention and treatment thereof has become urgent.

Currently, hair loss treatment preparations are largely classified into drugs, quasi-drugs, and cosmetics. Among ethical drugs that can be purchased only with a doctor's prescription, there is ‘Propecia’ developed and sold by Merck in the United States, and its main ingredient, Finasteride, was approved as a hair loss treatment by the US FDA in December 1997. Finasteride is a drug that inhibits the 5α-reductase enzyme that converts testosterone into dihydrotestosterone (DHT), and plays a role in making vellus hair grow into thick and long hair. This is effective in improving hair loss in the short term, but side effects such as erectile dysfunction, decreased sexual function, and enlarged male breasts have been reported.

As a drug that has been recognized for its safety and efficacy and can be purchased without a doctor's prescription, there is Minoxidil is, which was approved as the first topical hair loss treatment by the US FDA in December 1997. This drug has the effect of promoting hair growth by improving blood circulation and opening potassium channels, but it may cause local reactions such as itching and rashes, and tachycardia.

Therefore, in addition to the preparations known to have the effect of preventing hair loss and promoting hair sprouting and hair growth, it is necessary to develop a composition with secured safety and efficacy.

### SUMMARY OF THE INVENTION

### Technical Problem

In one aspect of the present disclosure, it is intended to provide a composition having hair sprouting or hair growth efficacy by including 3,4,5-trimethoxycinnamate thymol ester as an active ingredient.

### Technical Solution

In one aspect, the present disclosure provides a composition for promoting hair sprouting or hair growth, including 3,4,5-trimethoxycinnamate thymol ester or a stereoisomer, pharmaceutically acceptable salt, hydrate, or solvate thereof as an active ingredient.

### Advantageous Effects

In one aspect, the composition according to the present disclosure can be usefully used as a hair sprouting or hair growth promoting cosmetic composition, skin external application or pharmaceutical composition for the purpose of hair loss suppression and hair growth treatment.

### BRIEF DESCRIPTION OF THE DRAWINGS

The FIGURE is a result of confirming the hair growth efficacy of a composition according to one embodiment of the present disclosure by a Two-Cell Assemblage assay method.

### DETAILED DESCRIPTION OF THE INVENTION

Hereinafter, the present disclosure will be described in more detail by way of the following embodiments. However, the following embodiments are provided only for illustrative purposes to aid understanding of the present disclosure, and the scope and range of the present disclosure are not limited thereto.

In exemplary embodiments of the present disclosure, there is provided a composition for promoting hair sprouting or hair growth, including 3,4,5-trimethoxycinnamate thymol ester or a stereoisomer, pharmaceutically acceptable salt, hydrate, or solvate thereof as an active ingredient.

In another exemplary embodiment of the present disclosure, there is provided a method for promoting hair sprouting or hair growth, including administering a composition including 3,4,5-trimethoxycinnamate thymol ester or a stereoisomer, salt, hydrate, or solvate thereof as an active ingredient.

In still another exemplary embodiment of the present disclosure, there is provided a use of 3,4,5-trimethoxycinnamate thymol ester or a stereoisomer, salt, hydrate, or solvate thereof for preparing a composition for promoting hair sprouting or hair growth.

In yet another exemplary embodiment of the present disclosure, there is provided a non-therapeutic use of 3,4,5-trimethoxycinnamate thymol ester or a stereoisomer, salt, hydrate, or solvate thereof for promoting hair sprouting or hair growth.

In yet another exemplary embodiment of the present disclosure, there is provided 3,4,5-trimethoxycinnamate thymol ester or a stereoisomer, salt, hydrate, or sol vate thereof for promoting hair sprouting or hair growth.

In one embodiment, the 3,4,5-trimethoxycinnamate thymol ester is (5-methyl-2-propan-2-ylphenyl) (E)-3-(3,4,5-trimethoxyphenyl) prop-2-enoate, which may be represented by Chemical Formula 1 below:

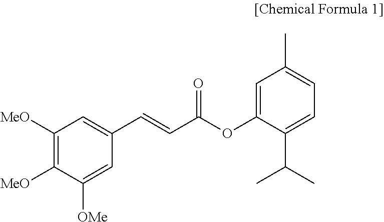 [SMILES: COc1cc(/C=C/C(=O)Oc2cc(C)ccc2C(C)C)cc(OC)c1OC]

As used herein, “isomer” includes in particular optical isomers (e.g., essentially pure enantiomers, essentially pure diastereomers or mixtures thereof), as well as conformation isomers (i.e., isomers which differ only in the angles of one or more chemical bonds), position isomers (in particular, tautomers) or geometric isomers (e.g., cis-trans isomers).

As used herein, the term “essentially pure”, for example, when used in connection with an enantiomer or diastereomer, means that a specific compound, for example an enantiomer or diastereomer, is present in an amount of at least about 90%, preferably at least about 95%, more preferably at least about 97% or at least about 98%, even more preferably at least about 99%, and even more preferably at least about 99.5% (w/w).

As used herein, the term “pharmaceutically acceptable” means that it can be or has been approved by the government or equivalent regulatory body as being suitable for use in animals, and more particularly, in humans, by avoiding significant toxic effects when used in conventional medicinal dosages, or is listed in a pharmacopoeia or recognized in other general pharmacopoeias.

As used herein, the term “pharmaceutically acceptable salt” means a salt according to one aspect of the present disclosure that is pharmaceutically acceptable and has the desired pharmacological activity of the parent compound. The salt may include (1) an acid addition salt formed of inorganic acid such as hydrochloric acid, hydrobromic acid, sulfuric acid, nitric acid, phosphoric acid, etc.; or formed of organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfate, gluconic acid, glutamic acid, hydroxynaphthoic acid, salicylic acid, stearic acid, muconic acid.; or (2) a salt formed when an acidic proton present in the parent compound is substituted.

As used herein, the term “hydrate” refers to a compound to which water is bound, and is a broad concept that includes an inclusion compound with no chemical bonding force between water and the compound.

As used herein, the term “solvate” refers to a high-order compound formed between a molecule or ion of a solute and a molecule or ion of a solvent.

In one embodiment, the composition may be used for promoting the growth of hair length.

In one embodiment, the concentration of the active ingredient may be 0.1 to 100 g/L based on the total volume of the composition.

For example, the concentration of the active ingredient may be 0.1 g/L or more, 0.5 g/L or more, 1 g/L or more, 5 g/L or more, 10 g/L or more, 20 g/L or more, 30 g/L or more, 40 g/L or more, 50 g/L or more, 60 g/L or more, 70 g/L or more, 80 g/L or more, or 90 g/L or more, and may be 100 g/L or less, 90 g/L or less, 80 g/L or less, 70 g/L or less, 60 g/L or less, 50 g/L or less, 40 g/L or less, 30 g/L or less, 20 g/L or less, 10 g/L or less, 5 g/L or less, 1 g/L or less, or 0.5 g/L or less, based on the total volume of the composition.

In one embodiment, the composition may be a cosmetic composition.

The external form of the cosmetic composition contains a cosmetically or dermatologically acceptable medium or base. It may be provided in any formulation suitable for topical application, for example, in the form of a solution, a gel, a solid, a pasty anhydrous product, an emulsion obtained by dispersing an oil phase in an aqueous phase, a suspension, a microemulsion, a microcapsule, a microgranule, or ionic (liposome) and nonionic vesicular dispersion, or in the form of a cream, a skin, a lotion, a powder, an ointment, a spray, or a concealer stick. These compositions can be prepared according to conventional methods in the art. The composition according to the present disclosure may also be used in the form of a foam or in the form of an aerosol composition further containing a compressed propellant.

The cosmetic composition according to one embodiment of the present disclosure is not particularly limited in its formulation, and may be formulated into cosmetics such as a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, and a body essence.

When the formulation of the cosmetic composition of the present disclosure is a paste, a cream, or a gel, animal fiber, plant fiber, wax, paraffin, starch, tragacanth, a cellulose derivative, polyethylene glycol, silicone, bentonite, silica, talc, or zinc oxide may be used as a carrier component.

When the formulation of the cosmetic composition of the present disclosure is a powder or spray, lactose, talc, silica, aluminum hydroxide, calcium silicate or polyamide powder may be used as a carrier component, and in particular, in the case of a spray, a propellant such as chlorofluorohydrocarbon, propane/butane or dimethyl ether may be additionally included.

When the formulation of the cosmetic composition of the present disclosure is a solution or an emulsion, a solvent, a solvating agent or an emulsifying agent is used as a carrier component, and the examples include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or a fatty acid ester of sorbitan.

When the formulation of the cosmetic composition of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used as a carrier component.

When the formulation of the cosmetic composition of the present disclosure is a surfactant-containing cleansing, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, fatty alcohol, fatty acid glyceride, fatty acid diethanolamide, vegetable oil, linoline derivative, ethoxylated glycerol fatty acid ester, etc. may be used as a carrier ingredient.

The cosmetic composition of the present disclosure may further include a functional additive and a component included in a general cosmetic composition in addition to the above-described active ingredients. The functional additive may include a component selected from the group consisting of water-soluble vitamins, oil-soluble vitamins, polymer peptides, polymer polysaccharides, sphingo lipids, and seaweed extract.

In the cosmetic composition of the present disclosure, in addition to the above functional additive, a component included in a general cosmetic composition may be blended as necessary. In addition, other blending components, such as oil and fat ingredients, moisturizers, emollients, surfactants, organic and inorganic pigments, organic powders, ultraviolet absorbers, preservatives, disinfectants, antioxidants, plant extracts, pH adjusters, alcohols, pigments, fragrances, blood circulation promoters, cooling agents, anhydrotics, purified water, etc., may be additionally included.

In one embodiment, the composition may be a skin external application composition.

The skin external application composition is a generic term that may include anything that is applied externally to the skin, and may include various types of cosmetics, pharmaceuticals, etc.

In one embodiment, the composition may be a food composition.

The food composition according to the present disclosure may be a liquid or solid formulation, and may be a tablet, a capsule, a soft capsule, a pills, a granule, a beverage (drinking agent), a diet bar, a chocolate, a caramel formulation, or a confectionery formulation, but the formulation is not particularly limited.

In addition to the above active ingredients, the food composition according to the present disclosure may further include excipients, sugars, flavors, pigments, fats, proteins, etc., as needed.

In one embodiment, the composition may be a pharmaceutical composition.

The pharmaceutical composition may be administered orally, parenterally, rectally, topically, transdermally, intravenously, intramuscularly, intraperitoneally, subcutaneously, etc. Formulations for oral administration may be tablets, pills, soft and hard capsules, granules, powders, fine granules, liquids, emulsions, or pellets, but are not limited thereto. Formulations for parenteral administration may be solutions, suspensions, emulsions, gels, injections, drops, suppositories, patches, or sprays, but are not limited thereto. The formulation may be easily prepared according to a conventional method in the art, and may further include a surfactant, an excipient, a hydrating agent, an emulsifying promoter, a suspending agent, a salt or buffer for osmotic pressure control, a coloring agent, a flavoring agent, a stabilizer, an antiseptic, a preservative, or other commonly used auxiliary agents.

In one embodiment, the 3,4,5-trimethoxycinnamate thymol ester or the stereoisomer, salt, hydrate, or solvate thereof may be administered at a dosage of 0.01 to 10 mg/kg/day.

For example, the dosage may be 0.01 mg/kg/day or more, 0.1 mg/kg/day or more, 1 mg/kg/day or more, 5 mg/kg/day or more, and may be 10 mg/kg/day or less, 5 mg/kg/day or less, 1 mg/kg/day or less, 0.1 mg/kg/day or less, or 0.05 mg/kg/day or less.

Hereinafter, the present disclosure will be described in more detail by way of the following examples. However, the following examples are provided only for illustrative purposes to aid understanding of the present disclosure, and the scope and range of the present disclosure are not limited thereto.

### Examples

### Preparation of 3,4,5-trimethoxycinnamate thymol ester

Thymol trimethoxycinnamate thymol ester with CAS number 504394-57-4 (Melasolv) was obtained from COSMANN Co., Ltd. (Hwaseong-si, Gyeonggi-do, Korea).

### Experimental Example—Evaluation of Hair Growth-Promoting Efficacy

The hair growth-promoting efficacy of 3,4,5-trimethoxycinnamate thymol ester (Melasolv) according to the concentration (10 ppm, 20 ppm) was evaluated using “Two-Cell Assemblage” Assay (“Two-Cell Assemblage” Assay: A Simple in vitro Method for Screening Hair Growth-Promoting Compounds) (Front Cell Dev Biol. 2020 Nov. 24; 8:581528).

The “Two-Cell Assemblage” Assay is an evaluation method that mimics the hair growth of actual hair follicles by co-culturing dermal papilla (DP) cells and outer root sheath (ORS) cells that constitute the hair follicle. That is, the length growth of the “Two-Cell Assemblage” structure has a mechanism similar to that of the actual hair follicle growth, and is being used as a new method of evaluating hair growth-promoting efficacy.

5,000 hair follicle dermal papilla cells were seeded on an ultra-low attachment 96-well plate to form 3D spheroids. After 24 hours, 5,000 outer root sheath cells (Kyungpook National University Industry-Academic Cooperation Foundation) were added together with the test substance to create a “Two-Cell assemblage” structure and cultured for 5 days under 37° C., 5% CO2 conditions. After culturing for 5 days, the length growth was measured using a microscope (OLYMPUS, SZX16) and shown in the Figure (* p<0.05, ** p<0.01, *** p<0.001 vs. control).

Referring to the Figure, when treated with Melasolv and cultured for 5 days with the “Two-Cell Assemblage” structure, a significant level of length growth-promoting efficacy, i.e., hair growth efficacy, can be confirmed.

What is claimed is:

1. A method for promoting hair sprouting or hair growth, comprising:
administering an effective amount of a composition comprising 3,4,5-trimethoxycinnamate thymol ester or a stereoisomer, pharmaceutically acceptable salt, hydrate, or solvate thereof as an active ingredient to a subject in need thereof.

2. The method according to claim 1, wherein the method promotes the growth of hair length.

3. The method according to claim 1, wherein the concentration of the active ingredient is 0.1 to 100 g/L based on the total volume of the composition.

4. The method according to claim 1, wherein the composition is a cosmetic composition.

5. The method according to claim 1, wherein the composition is a skin external application composition.

6. The method according to claim 1, wherein the composition is a food composition.

7. The method according to claim 1, wherein the composition is a pharmaceutical composition.

8. The method according to claim 1, wherein the 3.4.5-trimethoxycinnamate thymol ester or the stereoisomer, salt, hydrate, or solvate thereof is administered at a dosage of 0.01 to 10 mg/kg/day.
